(19) United States
(12) Patent Application Publication
(10) Pub. No.: US 2008/0287549 A1
(43) Pub. Date: Nov. 20, 2008

(54) METHODS AND MATERIALS FOR CONTROLLING RODENT POPULATIONS

(76) Inventors: Joseph S. Podolski, The Woodlands, TX (US); Ronald Wiehle, Houston, TX (US); Kuang Hsu, The Woodlands, TX (US)

Correspondence Address: HOWREY LLP - DC, C/O IP DOCKETING DEPARTMENT, 2941 FAIRVIEW PARK DR, SUITE 200, FALLS CHURCH, VA, 22042-2924, US

(21) Appl. No.: 11/750,197

(22) Filed: May 17, 2007

(51) Int. Cl. A61K 31/137; A61P 15/18

(52) U.S. Cl. 514/648

(57) ABSTRACT

The present invention relates to the use of compositions comprising clomiphene isomers for controlling rodent populations. The clomiphene isomers lower testosterone and/or suppress appetite in the rodent population.

### FIELD OF THE INVENTION

The present invention relates to compositions and methods for controlling rodent populations. More specifically, the present invention relates to the use of a composition comprising clomiphene, preferably enriched for cis-clomiphene, for controlling rodent populations.

### BACKGROUND

Rodents, such as rats and mice, have adapted to live in close proximity to humans. This co-habitation has resulted in a variety of economic and health problems for human populations, with an associated cost in the billions of dollars annually for the United States alone. Densely populated urban areas are especially vulnerable to rodents where rat populations have reached near-epidemic proportions. New York, for example, is host to an estimated 96 million rodents, outnumbering humans 12 to 1.

Rats pose a very serious health risk, carrying over thirty different diseases dangerous to humans including Weil's disease, typhus, salmonellosis, mycoplasmosis, bubonic plague, tuberculosis, rabies, foot-and-mouth disease, trichinosis and brucellosis. These diseases may be transmitted to humans from rat droppings, rat bites or from fleas that have acquired the disease from a rat. Hantavirus Pulmonary Syndrome may be transmitted to humans through the urine and droppings of mice and rats. Black rats are suspected to have had role in Black Death, an epidemic which killed at least 75 million people in Europe, the Middle East and Asia in the 14^{th} century.

Rodents also create a serious economic burden for human populations. For example, it is estimated that between ⅕ and ⅓ of the world's cumulative food production is eaten, spoiled or destroyed by rats. Third world countries are particularly vulnerable, as impoverished peoples often rely on a single crop for the main staple of their diet. Loss of a significant amount of grain crop to rats in such countries contributes to malnutrition. It is estimated that about 26% of electrical cable breaks and 18% of phone cable breaks result from rats chewing on the cables. Consequently, rats are estimated to be responsible for 25% of all fires of unknown origin.

A variety of measures have been used to control rat populations, generally employing the use of poisons and/or traps. However, current methods of rodent control are far from ideal. Poisons, for example, are not environmentally safe, as non-target species such as domesticated dogs and cats may consume the poison. Additionally, the poison may contaminate food and/or water supplies. Mechanical traps require a large investment in manpower and are quite expensive to set and maintain. Thus, there remains a need in the art for an effective alternative to traditional rodent control measures that overcomes the deficiencies of current rodent control methods.

### SUMMARY

The present invention is directed to compositions comprising clomiphene isomers useful for controlling rodent populations. In one of its aspects the invention is directed to compositions having active ingredients comprising 0% to 29% weight/weight (w/w) of cis-clomiphene and 100% to 71% w/w of trans-clomiphene or salts or solvates thereof. In another aspect, the invention is directed to compositions having active ingredients comprising 30% to 50% w/w of cis-clomiphene and 70% to 50% w/w of trans-clomiphene or salts or solvates thereof. In yet another aspect, the invention is directed to compositions having active ingredients comprising 0% to 49% w/w of trans-clomiphene and 100% to 51% w/w of cis-clomiphene or salts or solvates thereof. Among the preferred compositions of the present invention which contain both cis-clomiphene and trans-clomiphene are compositions wherein the ratio of trans-clomiphene and cis-clomiphene is less than or equal to 49/51. Most preferable according to the present invention is a composition comprising about 100% w/w of active ingredients of cis-clomiphene or a salt or solvate thereof. All compositions of the present invention may further comprise an edible carrier. Analogs of the trans- and cis-isomers of clomiphene such as those described in Ernst, et al. supra are also contemplated for use in all aspects of the present invention.

Also provided are uses of compositions of the present invention in methods of reducing testosterone in male rodents, methods of inducing hypophagia in rodents and methods of controlling rodent populations.

### BRIEF DESCRIPTION OF THE DRAWING

FIG. 1 shows the chemical structure of clomiphene citrate.

### DETAILED DESCRIPTION

The present invention provides methods and compositions useful for controlling rodent populations. The compositions comprise clomiphene isomers.

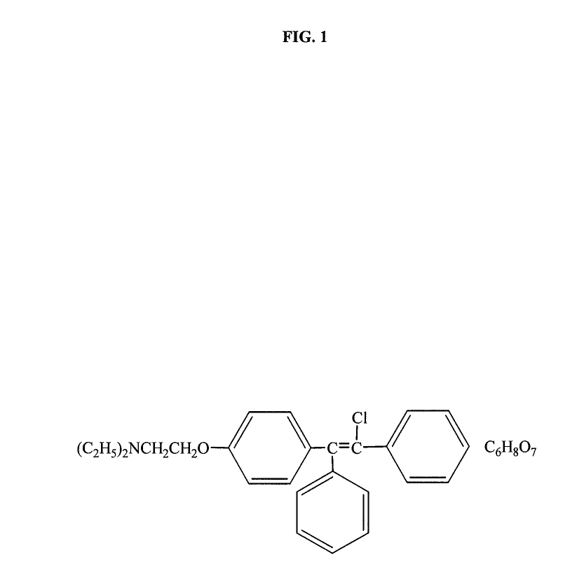

Ernst et al., J. Pharmaceut. Sci. 65:148 (1976), have shown that clomiphene (FIG. 1) is a mixture of two geometric isomers which they refer to as cis,-Z-, clomiphene (cis-clomiphene or zuclomiphene) and trans-,E-, clomiphene, (trans-clomiphene or enclomiphene). According to Ernst, et al. trans-clomiphene HCI has a melting point of 149° C.-150.5° C., while cis-clomiphene HCI has a melting point of 156.5° C.-158° C.

Clomiphene is an antiestrogen related to tamoxifen that blocks the normal estrogen feedback on the hypothalamus and subsequent negative feedback on the pituitary. Ernst et al. have noted that the trans-isomer is antiestrogenic (AE) while the cis-isomer is the more potent and more estrogenic form and has also been reported to have anti-estrogenic activity. The isomers are also reported to have different in vivo half-lives. The trans form is rapidly cleared, while the cis form clears slowly with significant blood level of the cis form detectable for in excess of one month following a single dose of clomiphene citrate.

Clomiphene citrate, a mixture of the cis- and trans-isomers, with the cis-isomer being present at about 30% to 50% (Merck Index), has been used for therapeutic intervention in men with low testosterone levels. Tenover et al., J. Clin. Endocrinol. Metab. 64:1103, (1987) and Tenover et al., J. Clin. Endocrinol. Metab. 64:1118 (1987) found increases in FSH, LH in both young and old men after treatment with clomiphene. They also found increases in free and total testosterone in men with young men showing significant increases. Similarly, in 1995, Guay et al. (Gray, et al., J. Clin. Endocrinol. Metab. 80:3546 (1995)) published a study in which they saw increase in LH, FSH, and testosterone after 2 months of clomiphene treatment.

The present invention arises in part from the unexpected finding that cis-clomiphene, and to a lesser extent trans-clomiphene, reduce circulating levels of testosterone in male rodents to very low levels, presumably through action on the hypothalamic-pituitary-testes axis. Of all mammals studied to date, only rats demonstrate a lowering of serum testosterone to near-castration levels in response to clomiphene and its isomers. Testosterone is the primary male androgen, playing a vital role in overall male health. Testosterone is essential to the development and maintenance of specific reproductive tissues (testes, prostate, epididymis, seminal vesicle, and penis) and male secondary sex characteristics. It plays a key role in libido and erectile function and is necessary for the initiation and maintenance of spermatogenesis. Reducing the level of testosterone in male rodents reduces their reproductive capacity and thus provides a non-lethal, cost-effective method of controlling rodent populations. Preferably, the level of testosterone in male rodents is decreased to castration or near-castration levels. Additionally, rats exhibit a rapid, marked hypophagia when given cis- or trans-clomiphene or mixtures of the two isomers.

Thus, the present invention is also directed to methods for decreasing serum testosterone levels in a rodent, comprising administering to the rodent, an effective amount of a composition according to the present invention, the composition having active ingredients comprising 0% to 29% w/w of cis-clomiphene and 100% to 71% w/w trans-clomiphene including any of their salts or solvates. Methods of the invention include those in which the administered compositions contain both isomers wherein the ratio of trans-clomiphene to cis-clomiphene is greater than 71/29. Methods of the invention also include those in which the composition administered to the rodent comprises about 100% w/w of trans-clomiphene.

The present invention is also directed to methods for decreasing serum testosterone levels in a rodent, comprising administering to the rodent, an effective amount of a composition according to the present invention, the composition having active ingredients comprising 30% to 50% w/w of cis-clomiphene and 70% to 50% w/w of trans-clomiphene, including any of their salts or solvates.

The present invention is also directed to methods for decreasing serum testosterone levels in a rodent, comprising administering to the rodent, an effective amount of a composition according to the present invention, the compositions having active ingredients comprising 0% to 49% w/we of trans-clomiphene and 100% to 51% w/w cis-clomiphene including any of their salts or solvates thereof. Among the preferred methods are those in which the administered compositions contain both isomers wherein the ratio of trans-clomiphene to cis-clomiphene is less than or equal to 49/51. Most preferred methods according to the instant invention include those in which the composition administered to the rodent comprises about 100% w/w of cis-clomiphene

The present invention also arises from the unexpected finding that cis-clomiphene and, to a lesser extent trans-clomiphene, exert hypophagic and anorexic effects on rats. Of all mammals studied to date, only rats responded to clomiphene and its isomers with a rapid reduction in body weight. This aspect of the invention is particularly useful where the aim is to reduce rodent appetite and ultimately reduce loss of food stocks intended for human consumption. It is anticipated that other animals will not demonstrate the same effects, rendering its action unusually safe.

Thus, the present invention is also directed to methods for suppressing appetite in a rodent, comprising administering to the rodent an effective amount of a composition according to the present invention, the compositions having active ingredients comprising 0% to 29% w/w of cis-clomiphene and 100% to 71% w/w trans-clomiphene including any salts or solvates thereof. Methods of the invention include those in which the administered compositions contain both isomers wherein the ratio of trans-clomiphene to cis-clomiphene is greater than or equal to 71/29. Methods of the invention also include those in which the composition administered to the rodent comprises about 100% w/w of trans-clomiphene.

The present invention is also directed to methods for suppressing appetite in a rodent, comprising administering to the rodent, an effective amount of a composition according to the present invention, the composition having active ingredients comprising 30% to 50% w/w of cis-clomiphene and 70% to 50% w/w of trans-clomiphene, including any of their salts or solvates.

The present invention is also directed to methods for suppressing appetite in a rodent, comprising administering to the rodent, an effective amount of a composition according to the present invention, the compositions having active ingredients comprising 0% to 49% w/w of trans-clomiphene and 100% to 51% w/w cis-clomiphene including any of their salts or solvates thereof. Among the preferred methods are those in which the administered compositions contain both isomers wherein the ratio of trans-clomiphene to cis-clomiphene is less than or equal to 49/51. Most preferred methods according to the instant invention include those in which the composition administered to the rodent comprises about 100% w/w of cis-clomiphene

The activities of clomiphene isomers in rats to suppress appetite and reduce testosterone levels lend themselves for use in rat populations where the aim is to reduce rat number and/or appetite and ultimately reduce the economic and health burdens on human populations. The cis-isomer is preferred due to stronger activities in reducing testosterone levels and appetite and also due its longer half-life.

The present invention thus provides a method for controlling a rodent population comprising the administration a composition comprising one isomer, preferably cis-clomiphene, or a predefined blend of the isomers of clomiphene as described below in an amount effective to suppress the appetite and/or reduce the testosterone levels of members of the rodent population. That these effects observed in response to compositions of the instant invention appear to be limited to rodents provides a distinct advantage over current methods of controlling rodent populations. The reversible nature of these effects is another advantage given that wild populations of rodents should rebound after cessation of intake.

In one embodiment of the present invention, an amount of a composition of the present invention is applied to a geographical area containing a rodent population which is to be controlled, wherein said amount is effective to induce hypophagic effects in rodents.

In another embodiment of the present invention, an amount of a composition of the present invention is applied to a geographical area containing a rodent population which is to be controlled, wherein said amount is effective to lower the testosterone levels of male rodents. Preferably, said amount is effective to lower the testosterone level of a male rodent to a level sufficient to inhibit the reproductive capacity of the rodent. More preferably, said amount is effective to lower the testosterone to castration level, defined as a testosterone level less than about 0.5 ng/ml.

Compositions of the present invention may be in the form of sustained release formulations prepared as described for example in U.S. Pat. No. 6,221,399, Japanese patent 4-312522, Meshali et al, Int. J. Phar. 89:177-181 (1993), Kharenko et al, Intern. Symp. Control Rel. Bioact. Mater. 22:232-233 (1995), WO 95/35093, Dangprasit et al., Drug. Devel. and Incl. Pharm. 21 (20):2323-2337 (1995); U.S. Pat. Nos. 6,143,353, 6,190,591, 6,096,338, 6,129,933, 6,126,969, 6,248,363 and other sustained release formulations well known in the art.

Compositions of the instant invention may be in the form of solids, such as tablets or filled capsules or liquids such as solutions suspensions, emulsions, elixirs or capsules filled with the same, all for oral use. Such compositions may comprise ingredients in conventional proportions. Such compositions preferably comprise an edible carrier which is palatable to members of the rodent population to be controlled. The edible carrier may be any substance palatable to the rodent, for example, grain products, flours, oils, sugar and the like. The active substance and edible carrier are mixed using known methods.

While compositions according to the present invention may be administered by the intravenous, subcutaneous, buccal, transmucusal, intrathecal, intradermal, intracisternal or other routes of administration, oral administration is the preferred route. Effective dosages may be determined by measuring serum testosterone levels after administration of the composition and altering the dosages, if necessary, to achieve a sufficient decrease in the serum testosterone levels to reduce reproductive capacity. Effective dosages may also be determined by measuring food or caloric intake prior to and subsequent to administration of the composition under identical conditions and altering the dosages, if necessary, to achieve a sufficient suppression of appetite.

Compositions according to the present invention may comprise trans-clomiphene at a dosage between 0.1 μg to about 20 mg (although the determination of optimal dosages is within the level of ordinary skill in the art). The composition may comprise cis-clomiphene at a dosage of about 0.1 μg, 0.2 μg, 0.3 μg, 0.4 μg, 0.5 μg, 0.6 μg, 0.7 μg, 0.8 μg, 0.9 μg, 1.0 μg, 1.5 μg, 2.0 μg, 2.5 μg, 3 μg, 3.5 μg, 4 μg, 4.5 μg, 5 μg, 5.5 μg, 6 μg, 6.5 μg, 7 μg, 7.5 μg, 8 μg, 8.5 μg, 9 μg, 9.5 μg, 0.01 mg, 0.02 mg, 0.03 mg, 0.04 mg, 0.05 mg, 0.10 mg, 0.15 mg, 0.20 mg, 0.25 mg, 0.30 mg, 0.35 mg, 0.40 mg, 0.45 mg, 0.50 mg, 0.55 mg, 0.60 mg, 0.65 mg, 0.70 mg, 0.75 mg, 0.80 mg, 0.85 mg, 0.90 mg, 0.95 mg, 1.00 mg, 1.10 mg, 1.20 mg, 1.30 mg, 1.40 mg, 1.50 mg, 1.60 mg, 1.70 mg, 1.80 mg, 1.90 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg or there between. The composition may comprise trans-clomiphene and cis-clomiphene at any ratio, however a ratio of about 49/51, 48/52, 47/53, 46/54, 45/55, 44/56, 43/57, 42/58, 41/59, 40/60, 39/61, 38/62, 37/63, 36/64, 35/65, 34/66, 33/67, 32/68, 31/69, 30/70, 29/71, 28/72, 27/73, 26/74, 25/75, 24/76, 23/77, 22/78, 21/79, 20/80, 19/81, 18/82, 17/83, 16/84, 15/85, 14/86, 13/87, 12/88, 11/89, 10/90, 9/91, 8/92, 7/93, 6/94, 5/95, 4/96, 3/97, 2/98, 1/99, 0.5/99.5, or there between is preferred. Analogs of the trans- and cis- isomers of clomiphene such as those described in Ernst, et al. supra are also useful in the practice of the present invention.

Compositions of the instant invention may be distributed randomly throughout a geographical area containing a rodent population to be controlled or may be positioned in fixed locations such as in bait houses. Where the rodent population to be controlled is a rat population, the distribution period is preferably 1-2 years.

The following Examples are meant to be illustrative of the invention and are not intended to limit the scope of the invention as set out is the appended claims.

### EXAMPLE 1

### Effects of Clomiphene and Its Isomers on Serum Testosterone and Body Weight in Rats

Male rats were given increasing doses (control=0 mg/kg, low=0.2 mg/kg, medium=2 mg/kg and high=20 mg/kg) of clomiphene, trans-clomiphene or cis-clomiphene for 7 consecutive days. Treatment was followed by a 7-day washout period. Serum testosterone levels and body weight were tested pre-treatment and post-treatment. Serum testosterone levels were analyzed by direct radioimmunoassay (RIA). Total serum testosterone did not differ among the treatment groups prior to treatment, as determined by analysis of variance (ANOVA) or by the Kruskal-Wallis test.

There were significant and unexpected decreases in serum testosterone and body weight among all treatment groups. See Table 1. Trans-clomiphene, cis-clomiphene and clomiphene all significantly and statistically lowered serum testosterone levels and caused a loss in body weight compared to the control group. Cis-clomiphene was most effective, reducing serum testosterone dramatically to near-castration levels in a dose-dependent manner (over 90% loss of total serum testosterone at the highest dose). Cis-clomiphene was also most effective in causing loss of body weight (loss of 75 g of body weight at the highest dose). There was a significant loss in both body weight and total serum testosterone with clomiphene that was dose-dependent. Trans-clomiphene resulted in a loss of body weight that was less severe than that observed following treatment with cis-clomiphene or clomiphene. Trans-clomiphene was also associated with a reduction in total serum testosterone that did not show an obvious dose-dependency.

TABLE 1

| | Average Decrease | |
|---|---|---|
| | in Serum | Average Loss |
| | Testosterone | of Body |
| Treatment Group | Level (%) | Weight (g) |
| Control (n = 9) | 19.6 | 2 |
| Low Trans-clomiphene (n = 8) | 71.7 | 34 |
| Med Trans-clomiphene (n = 9) | 68.3 | 40 |
| High Trans-clomiphene (n = 10) | 73 | 35 |
| Low Clomiphene (n = 9) | 65.2 | 24 |
| Med Clomiphene (n = 10) | 83.5 | 41 |
| High Clomiphene (n = 9) | 89.7 | 69 |
| Low Cis-clomiphene (n = 9) | 68.1 | 32 |
| Med Cis-clomiphene (n = 9) | 99.3 | 54 |
| High Cis-clomiphene (n = 10) | 99.6 | 75 |

### EXAMPLE 2

### Effect of Long-Term Administration of Low-Dosages of Trans-Clomiphene on Body Weight and Food Consumption in Rats

Twenty-eight weeks of a two-year study of the effects of low dosages of trans-clomiphene (0 mg/kg/day (control), 0.0125 mg/kg/day, 0.025 mg/kg/day and 0.05 mg/kg/day) on mean body weight and mean food consumption in age-matched male and female rats has been completed. For each group, n=60. Losses in body weight and decreases in food consumption in both male and female treatment groups (compared to control groups) have been observed in every treatment group. As the study has progressed, the losses in body weight and decreases in food consumption have become more pronounced. See Table 2 (M=male; F=female; Mean Body weight is in grams; Mean Food consumption is in grams/animal/day; and dosages are of trans-clomiphene in mg/kg/day). This study demonstrates that hypophagic effects of trans-clomiphene on both male and female rats are observed even at extremely low concentrations of the isomer.

TABLE 2

| | | | Week | Week | Week | Week | Week | Week | Week |
|---|---|---|---|---|---|---|---|---|---|
| Treatment Group | | Week 2 | 6 | 10 | 14 | 18 | 22 | 26 | 28 |
| M (control) | Mean Body | 348.4 | 465.9 | 522.8 | 562.2 | 597.9 | 631.3 | 661.5 | 667.5 |
| | weight | | | | | | | | |
| | Mean Food | 24.1 | 25.8 | 27.7 | 28.0 | 26.8 | 27.2 | 25 | 26.5 |
| | consumption | | | | | | | | |
| M | Mean Body | 355.5 | 467.0 | 519.0 | 552.1 | 572.5 | 597.8 | 619.6 | 620.2 |
| (0.0125) | weight | | | | | | | | |
| | Mean Food | 23.8 | 25.1 | 26.6 | 26.5 | 24.3 | 25.1 | 23.1 | 23.6 |
| | consumption | | | | | | | | |
| M | Mean Body | 347.4 | 447.8 | 498.0 | 527.8 | 543.3 | 566.3 | 590.0 | 594.2 |
| (0.025) | weight | | | | | | | | |
| | Mean Food | 24.3 | 23.8 | 26.9 | 25.4 | 23.6 | 24.0 | 23.4 | 22.6 |
| | consumption | | | | | | | | |
| M (0.05) | Mean Body | 335.2 | 414.1 | 451.7 | 480.5 | 496.9 | 518.8 | 536.8 | 542.9 |
| | weight | | | | | | | | |
| | Mean Food | 22.0 | 22.5 | 23.5 | 25.9 | 22.6 | 23.1 | 22.4 | 22.0 |
| | consumption | | | | | | | | |
| F | Mean Body | 228.5 | 282.6 | 298.6 | 313.6 | 323.4 | 339.1 | 353.2 | 355.3 |
| (control) | weight | | | | | | | | |
| | Mean Food | 17.9 | 18.6 | 19.3 | 20.0 | 18.3 | 19.1 | 17.6 | 18.3 |
| | consumption | | | | | | | | |
| F | Mean Body | 225.1 | 269.0 | 280.3 | 291.8 | 300.8 | 309.0 | 318.7 | 318.4 |
| (0.0125) | weight | | | | | | | | |
| | Mean Food | 17.1 | 17.4 | 17.5 | 20.0 | 17.5 | 17.8 | 16.9 | 16.4 |
| | consumption | | | | | | | | |
| F | Mean Body | 220.4 | 257.3 | 271.8 | 281.4 | 286.9 | 294.9 | 305.5 | 304.4 |
| (0.025) | weight | | | | | | | | |
| | Mean Food | 17.3 | 16.7 | 17.8 | 18.2 | 16.1 | 17.0 | 16.3 | 15.4 |
| | consumption | | | | | | | | |
| F (0.05) | Mean Body | 214.4 | 245.1 | 260.3 | 272.3 | 278.9 | 287.7 | 296.9 | 298.3 |
| | weight | | | | | | | | |
| | Mean Food | 16.0 | 15.8 | 17.4 | 19.0 | 16.4 | 17.5 | 16.3 | 15.3 |
| | consumption | | | | | | | | |

### EXAMPLE 3

### Method for Determining an Effective Amount of a Composition Comprising Cis-Clomiphene for Controlling Rodent Populations

Prior to administration of a composition comprising cis-clomiphene, blood samples are taken from subject male rodents and testosterone levels are measured using methodologies described for example in Matsumoto, et al. Clin. Endocrinol. Metab. 56; 720 (1983) (incorporated herein by reference). Sex hormone binding globulin (SHBG), both free and bound to testosterone, may also be measured as described for example in Tenover et al. J. Clin. Endocrinol. Metab. 65:1118 (1987) which describe measurement of SHBG by both a [³H] dihydrotestosterone saturation analysis and by radioimmunoassay. Non-SHBG-bound testosterone levels (bioavailable testosterone) are also measured for example according to Tenover et al. J. Clin. Endocrinol and Metab. 65:1118 (1987). See also Soderguard et al. J. Steroid Biochem 16:801 (1982) incorporated herein by reference.

Subject male rodents are given daily dosages of 1.5 mg/kg cis-clomiphene. Subject male rodents are monitored for testosterone levels such that the dosage amount may be adjusted to achieve levels of testosterone in the rodents sufficient to reduce reproductive capacity.

### EXAMPLE 4

### Preparation and Distribution of a Composition Comprising Cis-Clomiphene for Controlling Rodent Populations

Cis-clomiphene is dissolved in edible oil, preferably fish oil, and mixed with flour and water to create the bait compositions. Bait compositions are placed throughout a geographical area containing a rodent population to be controlled for a period time sufficient to decrease the rodent population. The bait compositions are distributed daily at least throughout the reproductive cycle of the rodent and preferably throughout the natural life span of the rodent.

1. A method for reducing the testosterone level of a male rodent comprising administering to the rodent an effective amount of a composition comprising cis-clomiphene and trans-clomiphene or analogs thereof or salts or solvates thereof.

2. The method of claim 1 wherein the ratio of trans-clomiphene to cis-clomiphene is less than or equal to 49/51.

3. The method of claim 1 wherein the composition consists essentially of an effective amount of cis-clomiphene or an analog thereof or a salt or solvate thereof.

4. The method of claim 3 wherein the rodent is a rat.

5. The method of claim 4 wherein the composition is applied in an amount effective to inhibit the reproductive capacity of the rat.

6. The method of claim 3 wherein cis-clomiphene is present in an amount from 0.1 μg to 5.0 mg.

7. A method for inducing hypophagia in a rodent comprising administering to the rodent an effective amount of a composition comprising cis-clomiphene and trans-clomiphene or analogs thereof or salts or solvates thereof.

8. The method of claim 7 wherein the ratio of trans-clomiphene to cis-clomiphene is less than or equal to 49/51.

9. The method of claim 7 wherein the composition consists essentially of an effective amount of cis-clomiphene or an analog thereof or a salt or solvate thereof.

10. The method of claim 9, wherein the rodent is a rat.

11. The method of claim 10, wherein the rat is a female rat.

12. The method of claim 7, wherein cis-clomiphene is present in an amount from 0.01 to 5.0 mg.

13. A method for controlling a rodent population, comprising the application of an effective amount of a composition comprising cis-clomiphene and trans-clomiphene or analogs thereof or salts or solvates thereof and an edible carrier, to an environment containing a rodent population which is to be controlled.

14. The method of claim 13 wherein the ratio of trans-clomiphene to cis-clomiphene is less than or equal to 49/51.

15. The method of claim 13 wherein the composition consists essentially of an effective amount of cis-clomiphene or an analog thereof or a salt or solvate thereof.

16. The method of claim 15 wherein the rodent population to be controlled is a rat population.

17. The method of claim 16, wherein the composition is applied in an amount effective to reduce the testosterone level of a male rat.

18. The method of claim 17 wherein the testosterone level is reduced to a level sufficient to inhibit the reproductive capacity of the rat.

19. The method of claim 16, wherein the composition is applied in an amount effective to induce hypophagia in a rat.

20. The method of claim 19, wherein said rat is a female rat.

21. The method of claim 16, wherein cis-clomiphene is present in an amount from 0.01 mg to 5.0 mg.
